(19) United States
(12) Patent Application Publication
(10) Pub. No.: US 2025/0292875 A1
(43) Pub. Date: Sep. 18, 2025

(54) METHOD AND SYSTEM OF PRE-TRAINING GRAPH NEURAL NETWORK FOR MOLECULAR GRAPHS

(71) Applicant: City University of Hong Kong, Hong Kong (CN)

(72) Inventors: Wei LIN, Hong Kong (CN); Chi Chung FUNG, Hong Kong (CN)

(21) Appl. No.: 18/607,650

(22) Filed: Mar. 18, 2024

(51) Int. Cl. G16C 20/70

(52) U.S. Cl. CPC G16C 20/70

(57) ABSTRACT

A computer-based method of pre-training a graph neural network for molecular graphs is provided. The method includes assigning a weight to each atom in a molecule of a molecular sample based at least on a number of atoms of each atomic type in the molecule, an atomic composition of the molecular sample is known, randomly masking a number of atoms in the molecule of the molecular sample based on the assigned weight of each atom, an atomic type having a higher number of atoms in the molecule has a lower probability of being masked, and pre-training the graph neural network using the masked molecular sample.

### FIELD OF INVENTION

The present invention relates broadly, but not exclusively, to a method and system of pre-training graph neural network for molecular graphs.

### BACKGROUND

Self-supervised pre-training has emerged as an effective paradigm in the realm of Natural Language Processing (NLP) due to the remarkable advancements of BERT series and GPT series. Specifically, BERT is designed to predict the randomly masked tokens of massive unlabeled texts by leveraging neighboring words, known as the Masked Language Model (MLM) task. Through pre-training, BERT masters the underlying grammatical rules and obtains better results in downstream tasks because of improved initialization parameters. Inspired by the success of this approach, researchers have extended similar masking strategies to diverse domains, including Computer Vision (CV) and AI4Science. MLM-style pre-training strategy is also utilized for pre-training Graph Neural Networks (GNNs) to extract chemical knowledge from large-scale unlabeled molecular graph datasets, with one pioneering work known as Attribute Masking (AttrMask).

However, MOLE-BERT, a two-stage GNN pre-training method for molecules, highlights a critical issue with AttrMask, namely the imbalanced distribution of atoms. For example, trace elements in a molecule may have little probability of being learned during pre-training if utilizing the random masking strategy. Consequently, the pre-trained model may predominantly focus on learning common element(s) present in the molecule and neglecting the relatively rare but potentially critical elements. To address the above issue, MOLE-BERT introduces a variant of Vector Quantised-Variational AutoEncoder (VQ-VAE) as an atom tokenizer, aiming to enlarge the atom vocabulary for obtaining various chemically meaningful discrete embeddings at the first stage. During the pre-training stage, GNNs are employed to predict the discrete tokens of the original graphs transformed by the trained VQ-VAE, noted as the Masked Atoms Modeling (MAM) task. Motivated by the pioneering Masked AutoEncoder (MAE) from CV, GraphMAE incorporates a GNN layer behind the backbone models to reconstruct the node features of graphs, as an improved version of AttrMask. However, these methods focus on augmenting the capacities of the backbone models by adding additional modules, thereby increasing the pre-training burden.

Thus, a need exists to provide a system and method to address at least some of the above-mentioned challenges.

Furthermore, other desirable features and characteristics will become apparent from the subsequent detailed description and the appended claims, taken in conjunction with the accompanying drawings and this background of the disclosure.

### SUMMARY

According to an aspect of the present invention, there is provided a computer-based method of pre-training a graph neural network for molecular graphs. The method includes assigning a weight to each atom in a molecule of a molecular sample based at least on a number of atoms of each atomic type in the molecule, wherein an atomic composition of the molecular sample is known, randomly masking a number of atoms in the molecule of the molecular sample based on the assigned weight of each atom, wherein an atomic type having a higher number of atoms in the molecule has a lower probability of being masked, and pre-training the graph neural network using the masked molecular sample.

Before assigning the weight to each atom, the method may include obtaining the molecular sample. Obtaining the molecular sample may include receiving a string representation of the molecular sample, and converting the string representation of the molecular sample into a molecular graph representation of the molecular sample.

In embodiments of the present invention, obtaining the molecular sample may further include determining the number of atoms of each atomic type in the molecule, and storing the number of atoms of each atomic type in the molecule in a digital memory.

In some embodiments, obtaining the molecular sample may further include receiving a 3-Dimensional (3D) x-ray crystal structure of the molecular sample, and transforming the 3D x-ray crystal structure of the molecular sample into the string representation of the molecular sample.

In embodiments of the present invention, the method may further include iteratively, generating a prediction of each of the number of masked atoms using the graph neural network, and comparing each of the predicted atoms with each corresponding atom in the molecule.

In embodiments of the present invention, assigning the weight to each atom in the molecule of the molecular sample may include storing each assigned weight in a digital memory.

In embodiments of the present invention, the weight of each atom in the molecule of the molecular sample may be determined based on the following equation:

$${w}_{a ( i )} = \frac{ln k ( {n}_{a ( i )} + 1 )}{{n}_{a ( i )}}$$

wherein wₐ₍ᵢ₎ represents the assigned weight of each atom in the molecule of the molecular sample, nₐ₍ᵢ₎ represents the number of atoms of each atomic type in the molecule, and k represents a hyperparameter.

In some embodiments, k may be a numerical value that is more than or equal to 0.8.

In embodiments of the present invention, after randomly masking the number of atoms in the molecule of the molecular sample, the method may further include determining a total number of atoms that are being masked, and determining a number of atoms of each atomic type in the molecule that are being masked based on the determined total number of atoms that are being masked.

In embodiments of the present invention, the number of atoms of each atomic type in the molecule that are being masked may be determined based on the following equation:

$${m}_{a ( i )} = M \times \frac{ln k ( {n}_{a ( i )} + 1 )}{{\sum }_{a ( i )} ln k ( {n}_{a ( i )} + 1 )}$$

wherein M represents the total number of atoms that are being masked, nₐ₍ᵢ₎ represents the number of atoms of each atomic type in the molecule, and k represents a hyperparameter.

In embodiments of the present invention, pre-training the graph neural network may include inputting the masked molecular sample into the graph neural network and performing self-supervised learning based on any one of the following self-supervised learning models: Attribute Masking (AttrMask), Graph Masked AutoEncoder (GraphMAE) or Masked Atoms Modeling (MAM).

In embodiments of the present invention, the trained graph neural network may be configured to perform prediction of an unknown molecular sample.

There is disclosed a computer-based system for pre-training a graph neural network for molecular graphs. The computer-based system includes at least one processor, a digital memory, and a non-transitory computer-readable storage medium coupled to the at least one processor and storing programming instructions for execution by the at least one processor, the programming instructions instruct the at least one processor to perform the method described above.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a table listing pre-training datasets used for pre-training a graph neural network.

FIG. 2 shows a schematic diagram of an exemplary system for pre-training a graph neural network for molecular graphs, in accordance with an embodiment.

FIG. 3 shows a flowchart illustrating a method of pre-training a graph neural network for molecular graphs, in accordance with an embodiment.

FIG. 4 shows another flowchart illustrating a method of pre-training a graph neural network for molecular graphs, in accordance with an embodiment.

FIG. 5 shows a flowchart illustrating a process framework of obtaining a molecular sample, in accordance with an embodiment.

FIG. 6 shows a flowchart illustrating a process framework of pre-training a graph neural network using a masked molecular sample, in accordance with an embodiment.

FIG. 7 shows a flowchart illustrating yet another method of pre-training a graph neural network for molecular graphs, in accordance with an embodiment.

FIG. 8 shows a bar chart illustrating an atomic composition of molecules in a pre-training dataset.

FIG. 9 shows molecular graphs of two example molecules.

FIG. 10 shows a line chart illustrating a relationship between a weight factor and a number of atoms of each atomic type in a molecule with varying values of a hyperparameter k, in accordance with an embodiment.

FIG. 11 shows a processing framework illustrating a comparison of a random masking strategy and a Weighted Random Masking strategy for Each Molecule (WMM), in accordance with an embodiment.

FIG. 12 shows a table illustrating a comparison between performances across different training models, in accordance with an embodiment.

FIG. 13A shows a line chart illustrating a comparison of obtained accuracies between Attribute Masking (AttrMask) and AttrMask (WMM) with varying values of the hyperparameter k, in accordance with an embodiment.

FIG. 13B shows a line chart illustrating a comparison of performances between Attribute Masking (AttrMask) and AttrMask (WMM) when the hyperparameter k is fixed at a value of 0.9, in accordance with an embodiment.

FIG. 14 shows a bar chart illustrating performances of GraphMAE (WMM) with varying values of the hyperparameter k, in accordance with an embodiment.

FIG. 15 shows a bar chart illustrating a comparison of performances between GraphMAE and GraphMAE (WMM) across different pre-training datasets, in accordance with an embodiment.

FIG. 16 shows a schematic diagram of an example of a computing device used to realise a system for pre-training a graph neural network for molecular graphs, in accordance with an embodiment.

### DETAILED DESCRIPTION

Embodiments of the present invention will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present disclosure also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a conventional computer will appear from the description below.

In addition, the present disclosure also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the present invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM, GPRS, 3G or 4G mobile telephone systems, as well as other wireless systems such as Bluetooth, ZigBee, Wi-Fi. The computer program when loaded and executed on such a computer effectively results in an apparatus that implements the steps of the preferred method.

The present invention may also be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC) or Field Programmable Gate Array (FPGA). Numerous other possibilities exist. Those skilled in the art will appreciate that the system can also be implemented as a combination of hardware and software modules.

In embodiments of the present invention, use of the term ‘server’ may mean a single computing device or at least a computer network of interconnected computing devices which operate together to perform a particular function. In other words, the server may be contained within a single hardware unit or be distributed among several or many different hardware.

The term “configured to” is used in the specification in connection with systems, apparatus, and computer program components. For a system of one or more computers to be configured to perform particular operations or actions means that the system has installed on IT software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions. For special-purpose logic circuitry to be configured to perform particular operations or actions means that the circuitry has electronic logic that performs the operations or actions.

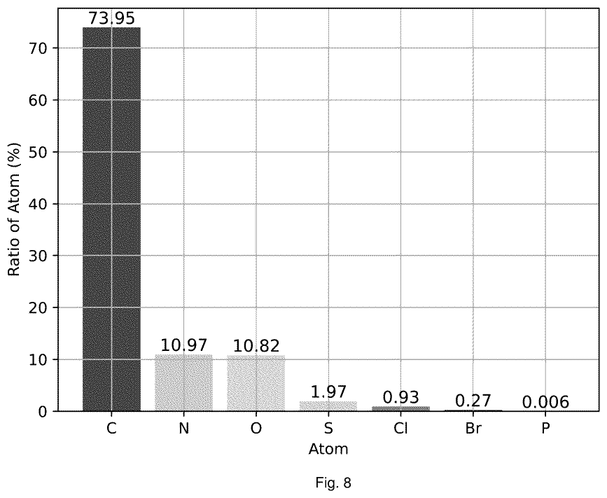

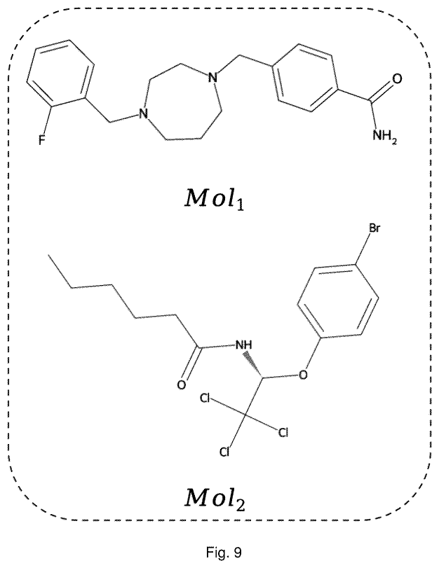

The present disclosure provides a weighted masking method/strategy from a data-centric perspective, aiming to mitigate the challenges arising from imbalanced atom ratios during pre-training. However, how to assign masking probabilities for different atom types is a crucial consideration. Some NLP researchers argue that high-frequency words such as definite articles tend to have less information contribution to the representations of sentence, but the situation may differ when considering molecules. For example, even though carbon atoms constitute the majority of total atoms, they can possess diverse meanings within various functional groups. Moreover, the distribution of Mol2 (see FIG. 9), is different from the atomic distribution in the pre-training dataset (as shown in FIG. 8), where the number of specific trace elements (or the lower-frequency atoms), e.g., chlorine, is more than some common elements (or the higher-frequency atoms) such as oxygen and nitrogen. To balance the above situations, a weighted masking strategy, also referred to as Weighted Random Masking strategy for Each Molecule (WMM) is provided in the present disclosure as a feasible data-centric solution. By adopting this strategy, the learning potential derived from mining molecules during pre-training can be maximized without the need for additional model parameters.

Experimental results have demonstrated that AttrMask with WMM outperforms AttrMask with random masking on more than half of the downstream datasets, yielding an overall performance improvement of 1.38%. Besides, the data-centric method (i.e., WMM) also surpasses model-centric enhancement methods such as MAM and GraphMAE in terms of average performance. Furthermore, the WMM is integrated with GraphMAE in experiments and it is observed that the capacity of GraphMAE is enhanced in over half of the datasets and achieves an overall average performance improvement of 0.89%, indicating the WMM can serve as a general framework for node-level pre-training strategies. Further, the trained graph neural network using WMM can serve as a backbone model to obtain molecular representations, which can be further used for drug design, drug discovery and ADMET (Absorption, Distribution, Metabolism, Excretion, Toxicity).

In conclusion, the present disclosure provides a data-centric perspective to analyze the functions of atoms in molecules and proposes a weighted masking strategy for pre-training molecular graphs. The results exemplify the versatility and effectiveness of WMM across various datasets and highlight its potential to enhance pre-training methodologies.

### Methods

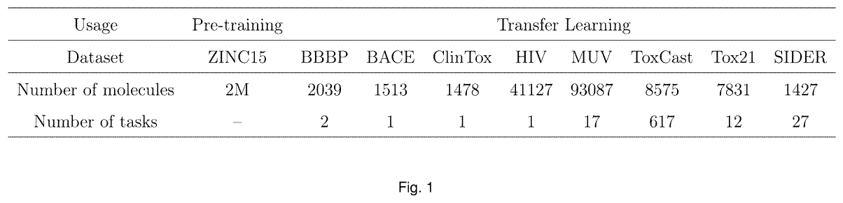

In the stage of pre-training, a dataset obtained from the ZINC15 database is utilized. Then, 8 datasets from MoleculeNet are adopted for transfer learning. The transfer learning centers around molecular property prediction. Details of these datasets are illustrated in FIG. 1. These datasets are typically in the SMILES (Simplified Molecular-Input Line Entry System) format. Hence, in order to convert these datasets into molecular graphs, RDKit, an open-source software, can be used to convert the datasets in the SMILES format into molecular graphs. Further, information on each atomic type present in the molecule can also be retrieved from the datasets in the SMILES format using the RDKit. The person skilled in the art would be aware of the procedure to convert these datasets into molecular graphs or retrieving the aforementioned information using RDKit. For example, molecule data received in the SMILES format can be denoted as “smiles” in the software, packages can be imported using “from rdkit. Chem import AllChem” and then change “smiles” into “mol=AllChem. MolFromSmiles (smiles)”. Then, information on the atom type of each atom can be obtained through “for atom in mol. GetAtoms( ): atom_type=atom.GetAtomicNum( )”. Further, it would be understood that the software used for the conversion and retrieval of information is not limited solely to RDKit and can encompass other software or application suitable for the intended purposes. The datasets can also be in formats other than SMILES and the person skilled in the art would be familiar with the procedures to perform such conversion and retrieval of information using other software or application.

Optionally, a 3-Dimensional (3D) x-ray crystal structure of a molecule can be obtained and converted/transformed into the SMILES format, which can be further converted/transformed into molecular graphs using the above-described technique. The person skilled in the art would recognise the required procedure for performing such conversion, which will not be described in detail in the present disclosure.

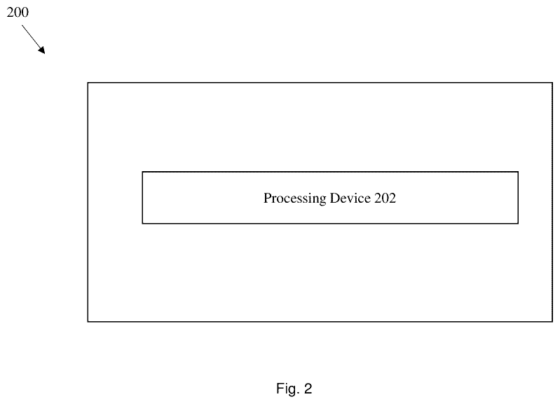

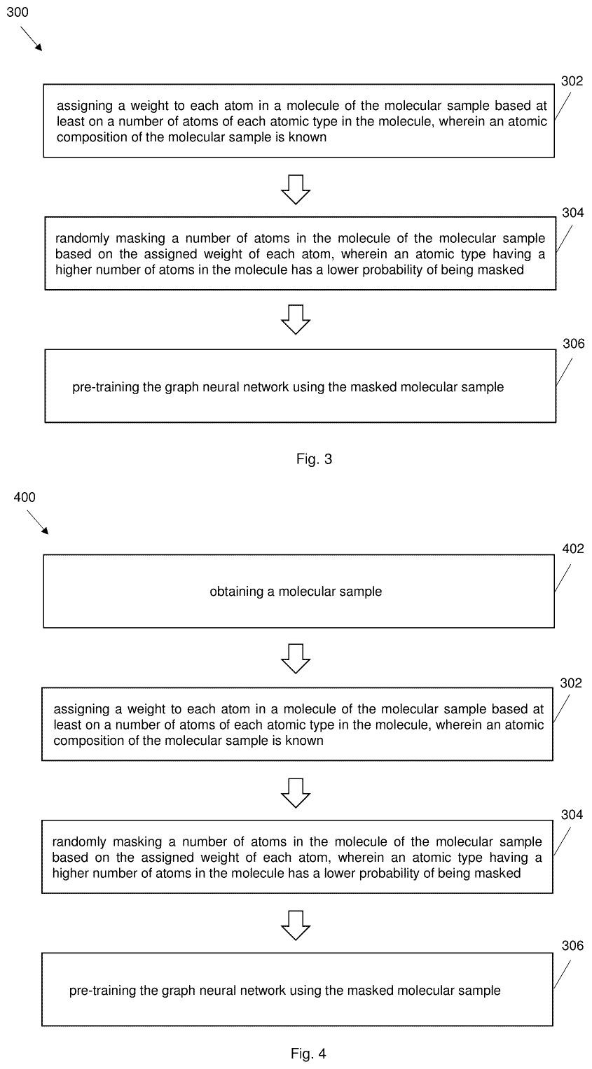

Embodiments of the present invention provide a method of pre-training a graph neural network for molecular graphs. The method can be implemented with system 200 shown in FIG. 2, which shows a schematic diagram of the system 200 for pretraining a graph neural network for molecular graphs. The system 200 can include a processing device 202. With reference to FIG. 3, the method 300 includes the following steps.

Step 302: assigning a weight to each atom in a molecule of the molecular sample based at least on a number of atoms of each atomic type in the molecule, wherein an atomic composition of the molecular sample is known.

Step 304: randomly masking a number of atoms in the molecule of the molecular sample based on the assigned weight of each atom, wherein an atomic type having a higher number of atoms in the molecule has a lower probability of being masked.

Step 306: pre-training the graph neural network using the masked molecular sample.

The weight of each atom in the molecule of the molecular sample can be determined based on Eq. (1) as defined below. In some embodiments, k is a numerical value that is more than or equal to 0.8.

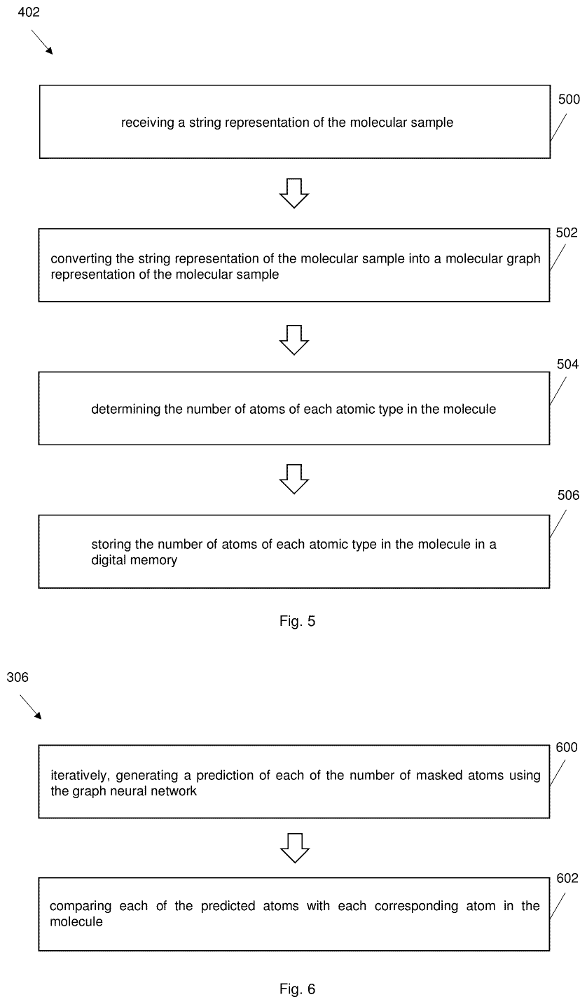

As shown in FIG. 4, the method 400 can further include a step 402 of obtaining a molecular sample. Further, as shown in FIG. 5, the step 402 of obtaining a molecular sample can include a step 500 of receiving a string representation of the molecular sample, a step 502 of converting the string representation of the molecular sample into a molecular graph representation of the molecular sample, and a step 504 of determining the number of atoms of each atomic type in the molecule. In some embodiments, the step 402 of obtaining a molecular sample can further include a step 506 of storing the number of atoms of each atomic type in the molecule in a digital memory. It should be understood that the string representation of the molecular sample can be in the form of, but not limited to, SMILES format, etc.

Optionally, the step 402 of obtaining a molecular sample can include receiving a 3-Dimensional (3D) x-ray crystal structure of the molecular sample, and transforming the 3D x-ray crystal structure of the molecular sample into the string representation of the molecular sample.

As shown in FIG. 6, the step 306 of pre-training the graph neural network using the masked molecular sample can include a step 600 of iteratively, generating a prediction of each of the number of masked atoms using the graph neural network and a step 602 of comparing each of the predicted atoms with each corresponding atom in the molecule.

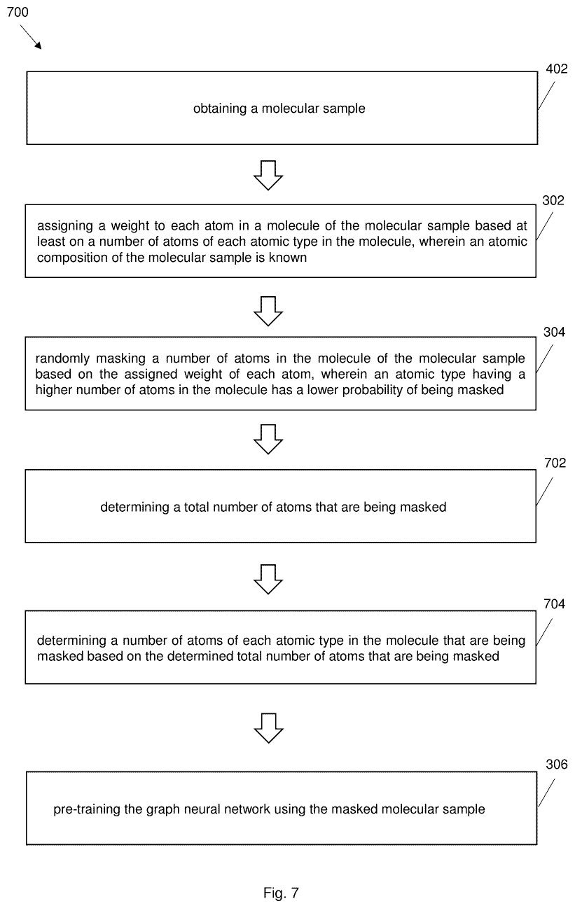

Additionally, as shown in FIG. 7, the method 700 can include a step 702 of determining a total number of atoms that are being masked and a step 704 of determining a number of atoms of each atomic type in the molecule that are being masked based on the determined total number of atoms that are being masked. The total number of atoms of each atomic type in the molecule that are being masked can be determined based on Eq. (2) as defined below.

Optionally, the step 302 of assigning the weight to each atom in the molecule of the molecular sample further includes storing each assigned weight in a digital memory. The step 306 of pre-training a graph neural network can include inputting the masked molecular sample into the graph neural network and performing self-supervised learning based on any one of the following self-supervised learning models: Attribute Masking (AttrMask), Graph Masked AutoEncoder (GraphMAE) or Masked Atoms Modeling (MAM). Further, the trained graph neural network can be configured to perform prediction of an unknown molecular sample.

FIG. 8 shows a visual representation illustrating the imbalanced nature of overall atom distribution in the pre-training dataset. Notably, carbon, oxygen, and nitrogen atoms account for approximately 96% of the total atoms. However, a significant portion of trace elements constitute the rest, resulting in an inherently imbalanced distribution. Besides, taking two molecules from the pre-training dataset as examples, it can be seen that the atom distributions of the two molecules are diverse and inconsistent with the overall atom distribution, as depicted in FIG. 9. Specifically, Mol₂ contains more chlorine, which is typically considered the trace element based on the overall distribution, than some common elements such as oxygen and nitrogen. Further, Phenyl groups, commonly found in the drug molecules, contribute to carbon atoms being the predominant atoms, as shown in FIG. 9. From a chemical standpoint, carbon atoms within phenyl groups fulfill similar roles within the molecule. Consequently, if the majority of masked atoms consist of carbon atoms from phenyl groups, the pre-trained models will gain limited information from the molecules. In addition, it is observed that similar representations among chlorine atoms in Mol₂, suggesting that high-frequency atoms (e.g., carbon atoms) are more likely to possess redundant information within this set of atoms. Conversely, the higher-frequency atoms tend to exhibit more semantic representation. For instance, some carbon atoms in Mol₂ belong to phenyl groups, while some belong to the carbonyl group. When considering trace elements, it is noted that substituting bromine with chlorine in Mol₂ is acceptable since both elements belong to the halogen group and share similar properties. This similarity poses a challenge for pre-trained models to effectively differentiate between them. On a separate note, masking the fluorine in Mol₁ has the potential of negatively impact the overall semantics of the molecule.

Based on the aforementioned analysis, one of the objectives that the present invention aims to achieve is to mask more rare elements to tackle the problem posed by the imbalanced distribution. However, it is important to ensure that the semantic representation of the molecule remains intact. Therefore, a criterion is established for the weighted masking strategy. In particular, a lower weight is assigned for higher-frequency atoms compared to lower-frequency atoms. Nevertheless, despite the lower weight, a larger number of higher-frequency atoms are subjected to masking compared to lower-frequency atoms. In light of the above-mentioned situations, the present disclosure proposes a data-centric solution, i.e., WMM. In this masking strategy, the random distribution of atom masking is replaced with a more refined approach. In some embodiments, atoms can be masked based on the following weighting factor (or weight), wₐ₍ᵢ₎, shown in Eq. (1):

$${w}_{a ( i )} = \frac{ln k ( {n}_{a ( i )} + 1 )}{{n}_{a ( i )}} \qquad - Eq . ( 1 )$$

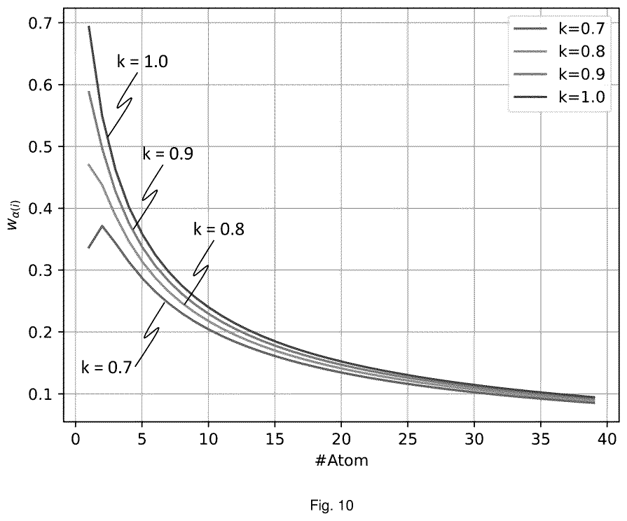

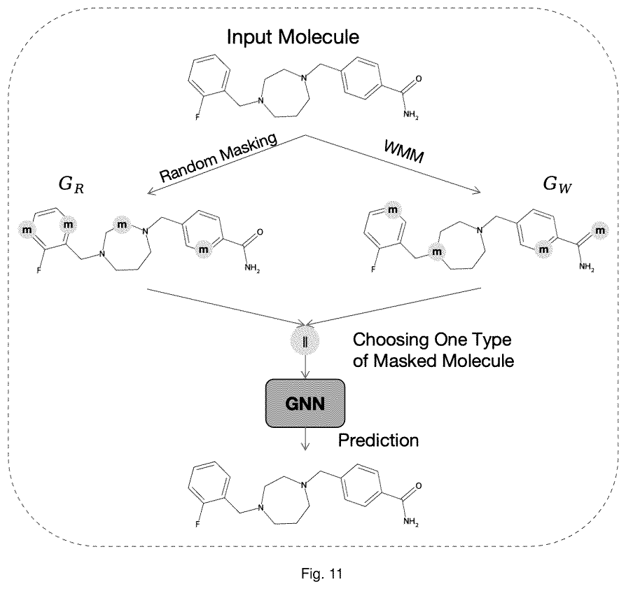

For any molecule Mol= (a₁, a₂, . . . , aᵢ, . . . , a_{N}) having N atoms, the objective is to determine M atoms to be masked for pre-training. For example, the M atoms to be masked can be determined based on both N atoms and a masking ratio (which is discussed in the later sections of the present disclosure). First of all, the probability of a particular atom being masked is proportional to the weight, while the weight is based on the number of atoms belonging to the same atomic type in this molecule, denoted by nₐ₍ᵢ₎, where α(i)∈{C, N, O, S, F, Cl, . . . }. Further, the form of Eq. (1) allows easy incorporation of a hyperparameter k to control the values of wₐ₍ᵢ₎. FIG. 10 illustrates that when nₐ₍ᵢ₎ is large, wₐ₍ᵢ₎ remains relatively unchanged as k increases. However, the trend differs when nₐ₍ᵢ₎ is small, indicating that increasing the hyperparameter k leads to higher probabilities of masking rare elements. Additionally, based on the established criterion mentioned above, lower masking probabilities are assigned for higher-frequency atoms within each molecule, so k is typically set to >0.8 to achieve this.

It should be understood that the weight factor is not limited to be determined using Eq. (1) but can encompass other schemes suitable for the intended purpose. For example, the weighting factor can also be determined using:

$${w}_{a ( i )} = ln \frac{k ( {n}_{a ( i )} + 1 )}{{n}_{a ( i )}} .$$

Additionally, after performing the weighted random masking, the number of masked atoms of each atomic type in the molecule, mᵢ, can be determined by Eq. (2) below:

$${m}_{a ( i )} = M \times \frac{ln k ( {n}_{a ( i )} + 1 )}{{\sum }_{a ( i )} ln k ( {n}_{a ( i )} + 1 )} \qquad - Eq . ( 2 )$$

The term In k(nₐ₍ᵢ₎+1) is a monotone increasing function when k>0.8, implying that the greater the value of nₐ₍ᵢ₎, the higher the number of masked atoms. A vivid explanation of the masking method is provided as follows with the aid of FIG. 11. Firstly, the task is to predict the masked atoms and Mol₁ (see FIG. 9) is used as the input molecule for this example. As shown in FIG. 11, if random masking is applied alone, there is a higher likelihood of masking carbon atoms (see G_{R}). Since these carbon atoms exhibit similar semantic representations, the pre-trained models would derive limited benefit from them. However, the comprehensive information about the atoms obtained from G_{W}, i.e., with WMM applied, would encourage the pre-trained models to learn more effectively.

As shown in FIG. 11, upon masking the molecule, the masked molecule can be input to a GNN. It should be understood that the GNN can encompass any models that are suitable for its intended purposes. In an example embodiment, consider an undirected molecular graph G=(V, E), whose node attributes Xᵥ for v∈V and edge attributes eᵤᵥ for (u, v)∈E. Let hᵥ^{(j)} denote the representation of node v at the j-th layer of GNN and N₍ᵥ₎ is a set neighbor of node v. The forward propagation formula from the (j−1)-th layer to the j-th layer can be expressed as follows:

$${h}_{v}^{( j )} = {COMBINE}^{( k )} ( {h}_{v}^{( j - 1 )} , {AGGREGATE}^{( j )} ( \{ {h}_{v}^{( j - 1 )} , {h}_{u}^{( j - 1 )} , {e}_{uv} ) : u ∈ 𝒩 ( v ) \} ) ) \qquad - Eq . ( 3 )$$

where AGGREGATE (⋅) is the aggregation function of the neighborhood information and COMBINE (⋅) means combining the information of neighbors and node v together. Besides, hᵥ⁽⁰⁾ (is initialized, i.e., hᵥ⁽⁰⁾=Xᵥ. Here, Xᵥ is the input features of the masked molecules.

To obtain the entire graph's representation h_{G}, the READOUT function pools node features from the final iteration J as expressed in Eq. (4) below:

$${h}_{G} = READOUT ( \{ {h}_{v}^{( j )} \vert v ∈ V \} ) \qquad - Eq . ( 4 )$$

where READOUT is a permutation-invariant function, such as averaging or a more sophisticated graph-level pooling function.

### Results

For experimental purposes, a 5-layer Graph Isomorphism Network (GIN) with a dimension of 300 is employed. Further, mean pooling is chosen as the readout function. During the pre-training stage, the GNN is pre-trained for 100 epochs with a batch size of 256 and a dropout rate of 0.0. For the transfer learning stage, the models are trained for 100 epochs using a batch size of 32 and a dropout rate of 0.5. The final test results are determined by the best validation results. To ensure out-of-distribution predictions, scaffold splitting is used to partition the molecules according to their structures. For both stages, Adam is adopted as an optimizer with a learning rate of 10-3. The train/validation/test sets are split in 80%: 10%: 10% ratio. To ensure robustness and fairness in the results, the pre-trained models are fine-tuned with 10different random seeds (0-9) and the results are averaged across the 10 runs to obtain the final performance for each dataset.

In this study, the mask ratio is maintained at 15%, 25%, and 15% for AttrMask, Graph-MAE, and MAM, respectively, as per their original experimental settings. Since MAM is designed to tackle the problem of imbalanced atom distribution, the method is only tested with AttrMask and GraphMAE in this study. It should be noted that pre-training tasks with the proposed masking strategy are denoted as AttrMask (WMM) and GraphMAE (WMM) for clarity. However, the pre-training methods with random masking will not be explicitly annotated. Additionally, the experiments are conducted with k∈[0.8, 1.2] to determine how the k value affects the performance of AttrMask. Then, only k∈[0.8, 1.0] is employed for GraphMAE to assess the generality of the proposed approach based on the results of AttrMask.

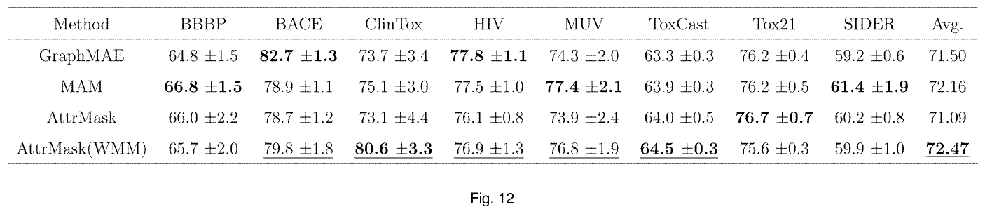

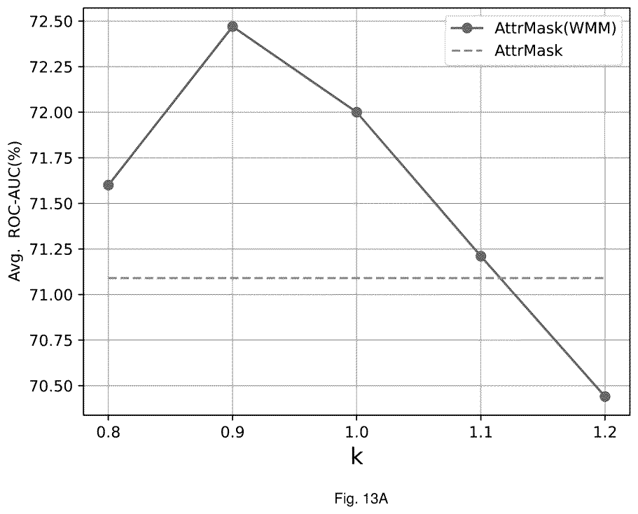

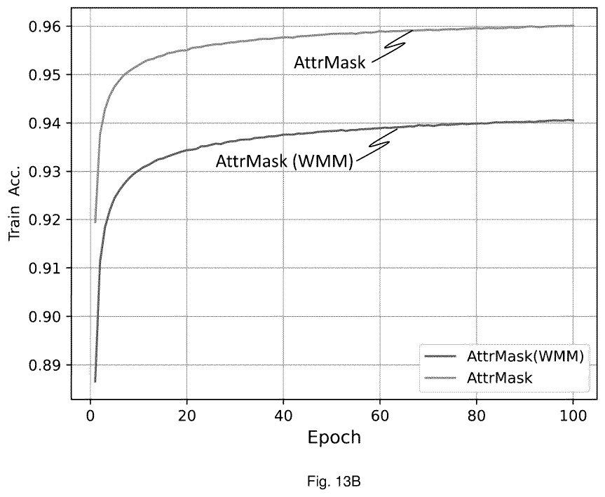

According to the results presented in FIG. 12, AttrMask with the proposed WMM strategy (i.e., AttrMask (WMM)) outperforms AttrMask with the random masking strategy on the majority of datasets (5 out of 8) in molecular prediction tasks, with a significant improvement on ClinTox and MUV. Although AttrMask (WMM) shows slightly lower performance on some datasets, the negative impacts on BBBP and SIDER can be considered negligible. Furthermore, AttrMask (WMM) achieves a notable improvement of 1.38% in overall performance. Remarkably, AttrMask (WMM) even exhibits superior performance in terms of overall performance compared with other model-centric improvement methods such as MAM and GraphMAE, despite the varying strengths of different pre-training methods in different datasets. Further, the results depicted in FIG. 13A indicate that the proposed WMM can obtain better results across a broad range of hyperparameters k, ranging from 0.8 to 1.1. However, it is worth noting that the WMM does lead to a degradation in the overall performance of AttrMask when k=1.2. This aligns with the hypothesis that masking too many trace elements may damage the molecular information and further result in worse performance. FIG. 13B demonstrates that the pre-training accuracy of AttrMask with the random masking strategy rapidly converges to approximately 96% and has less improvement compared to AttrMask (WMM). These observations indicate that AttrMask may be prone to overfitting to high-frequency atoms, such as carbon atoms, thereby limiting the pre-trained model to obtain adequate chemical knowledge. In contrast, the proposed WMM strategy achieves greater accuracy improvement while pre-training but only converges to approximately 94%, which indicates that the pre-trained models face a more difficult task and learn more knowledge from the pre-training task. This may account for the improved performance of the downstream tasks.

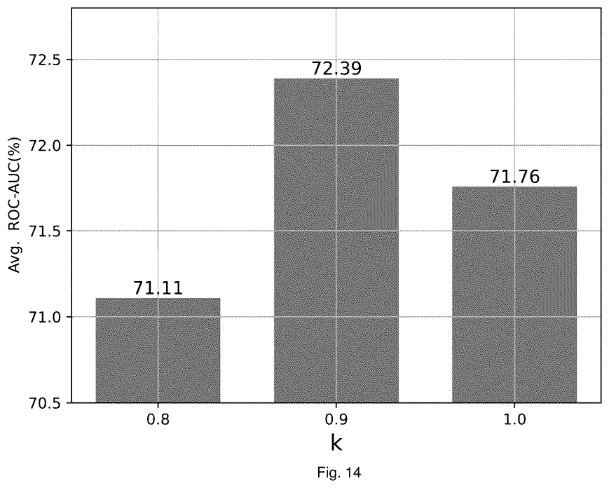

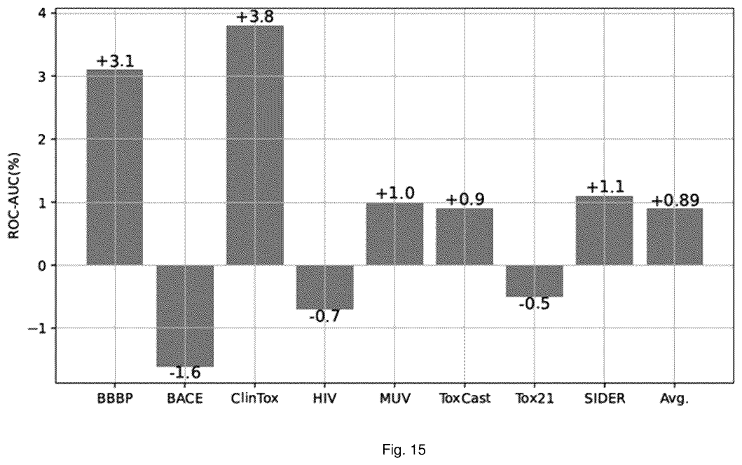

Moreover, this data-centric approach can be extended to a generative pre-training strategy noted as GraphMAE. Results have revealed that the task attains optimal outcomes when k=0.9, matching the performance of AttrMask, as illustrated in FIG. 14. FIG. 15 displays results that demonstrate the superiority of the masking strategy over the original GraphMAE on the majority of downstream datasets (5 out of 8), leading to a noticeable improvement of 0.89%. These findings indicate the efficacy and versatility of the masking strategy across various pre-training strategies.

### Discussion

However, in molecule pre-training, the focus is on the atoms within each molecule rather than the molecules themselves. Besides, up-sampling or data augmentation will destroy the semantics of molecules. Thus, down-sampling is a viable option for addressing this issue, but the crucial question remains: how to determine the masking probabilities for each atom type? The results, as shown in FIG. 7A, validate the former criteria that the performance of the pre-trained models is improved only when the atoms are masked in an appropriate way. Similarly, in the field of NLP, researchers have recognized the importance of informative low-frequency words, although the presence of noise in texts remains a challenge. To tackle the problem, researchers propose Prompt learning which encourages pre-trained models to predict some manually selected words to further unleash their potential. The weighted masking strategy shares some similarities with this approach, but it can also be utilized for pre-training since molecular inputs are generally cleaner compared to texts.

The present disclosure provides a method/strategy that is primarily focused on the assignment of varying weights to different atom types, assuming that each atom within a specific type contributes equally to the molecule. However, it is vital to note that each atom type can have multiple characteristics. For instance, carbon atoms can form single bonds, double bonds, or triple bonds, each with distinct properties. Taking into account these additional factors may potentially provide more benefits to pre-trained models, leading to improved performance in the pre-training phase. Additionally, it is possible to utilize SMILE (Simplified Molecular-Input Line Entry System) sequences as the inputs of pre-training., Furthermore, this method can also be extended to the field of biology for pre-training protein sequences. This extension is justified by the fact that an imbalanced distribution is an inherent characteristic of the natural world in biological systems. Besides, truncating protein sequences is unlikely to have a substantial impact on the overall sequence representations. This is primarily due to the fact that protein sequences are typically much larger in size compared to molecules, and thus, the removal of a portion of the sequence through truncation is less likely to significantly alter its overall representation.

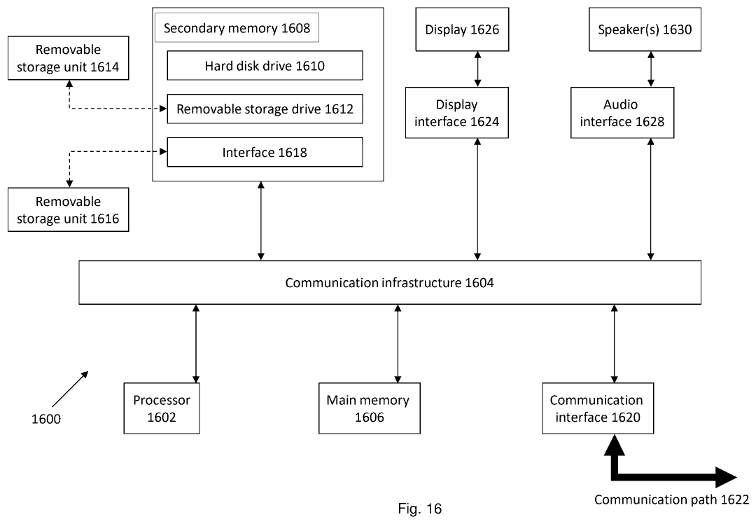

As shown in FIG. 16, an example computing device 1600 includes a processor 1602 for executing software routines. Although a single processor is shown for the sake of clarity, the computing device 1600 may also include a multi-processor system. The processor 1602 is connected to a communication infrastructure 1604 for communication with other components of the computing device 1600. The communication infrastructure 1604 may include, for example, a communications bus, cross-bar, or network.

The computing device 1600 further includes a main memory 1606, such as a random access memory (RAM), and a secondary memory 1608. The secondary memory 1608 may include, for example, a hard disk drive 1610 and/or a removable storage drive 1612, which may include a floppy disk drive, a magnetic tape drive, an optical disk drive, or the like. The removable storage drive 1612 reads from and/or writes to a removable storage unit 1614 in a well-known manner. The removable storage unit 1614 may include a floppy disk, magnetic tape, optical disk, or the like, which is read by and written to by removable storage drive 1612. As will be appreciated by persons skilled in the relevant art(s), the removable storage unit 1614 includes a computer readable storage medium having stored therein computer executable program code instructions and/or data.

In an alternative implementation, the secondary memory 1608 may additionally or alternatively include other similar means for allowing computer programs or other instructions to be loaded into the computing device 1600. Such means can include, for example, a removable storage unit 1616 and an interface 1618. Examples of a removable storage unit 1616 and interface 1618 include a program cartridge and cartridge interface (such as that found in video game console devices), a removable memory chip (such as an EPROM or PROM) and associated socket, and other removable storage units 1616 and interfaces 1618 which allow software and data to be transferred from the removable storage unit 1616 to the computer system 1600.

The computing device 1600 also includes at least one communication interface 1620. The communication interface 1620 allows software and data to be transferred between computing device 1600 and external devices via a communication path 1622. In various embodiments of the inventions, the communication interface 1620 permits data to be transferred between the computing device 1600 and a data communication network, such as a public data or private data communication network. The communication interface 1620 may be used to exchange data between different computing devices 1600 which such computing devices 1600 form part an interconnected computer network. Examples of a communication interface 1620 can include a modem, a network interface (such as an Ethernet card), a communication port, an antenna with associated circuitry and the like. The communication interface 1620 may be wired or may be wireless. Software and data transferred via the communication interface 1620 are in the form of signals which can be electronic, electromagnetic, optical or other signals capable of being received by communication interface 1620. These signals are provided to the communication interface via the communication path 1622.

As shown in FIG. 16, the computing device 1600 further includes a display interface 1624 which performs operations for rendering images to an associated display 1626 and an audio interface 1628 for performing operations for playing audio content via associated speaker(s) 1630.

As used herein, the term “computer program product” may refer, in part, to removable storage unit 1614, removable storage unit 1616, a hard disk installed in hard disk drive 1610, or a carrier wave carrying software over communication path 1622 (wireless link or cable) to communication interface 1620. Computer readable storage media refers to any non-transitory tangible storage medium that provides recorded instructions and/or data to the computing device 1600 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 1600. Examples of transitory or non-tangible computer readable transmission media that may also participate in the provision of software, application programs, instructions and/or data to the computing device 1600 include radio or infra-red transmission channels as well as a network connection to another computer or networked device, and the Internet or Intranets including e-mail transmissions and information recorded on Websites and the like.

The computer programs (also called computer program code) are stored in main memory 1606 and/or secondary memory 1608. Computer programs can also be received via the communication interface 1620. Such computer programs, when executed, enable the computing device 1600 to perform one or more features of embodiments discussed herein. In various embodiments, the computer programs, when executed, enable the processor 1602 to perform features of the above-described embodiments. Accordingly, such computer programs represent controllers of the computer system 1600.

Software may be stored in a computer program product and loaded into the computing device 1600 using the removable storage drive 1612, the hard disk drive 1610, or the interface 1618. Alternatively, the computer program product may be downloaded to the computer system 1600 over the communications path 1622. The software, when executed by the processor 1602, causes the computing device 1600 to perform functions of embodiments described herein.

It is to be understood that the embodiment of FIG. 16 is presented merely by way of example. Therefore, in some embodiments one or more features of the computing device 1600 may be omitted. Also, in some embodiments, one or more features of the computing device 1600 may be combined together. Additionally, in some embodiments, one or more features of the computing device 1600 may be split into one or more component parts.

It will be appreciated that the elements illustrated in FIG. 16 function to provide means for performing the various functions and operations of the servers as described in the above embodiments.

When the computing device 1600 is configured to realise the system 200 to pre-train a graph neural network for molecular graphs, the system 200 can have a non-transitory computer readable medium having stored thereon an application which when executed causes system 200 to perform a method with the following steps: assigning a weight to each atom in a molecule of a molecular sample based at least on a number of atoms of each atomic type in the molecule, wherein an atomic composition of the molecular sample is known; randomly masking a number of atoms in the molecule of the molecular sample based on the assigned weight of each atom, wherein an atomic type having a higher number of atoms in the molecule has a lower probability of being masked; and pre-training the graph neural network using the masked molecular sample.

The weight of each atom in the molecule of the molecular sample can be determined based on Eq. (1), wherein k can be a numerical value that is more than or equal to 0.8.

The method can also include a step of obtaining the molecular sample, wherein the step of obtaining the molecular sample can include receiving a string representation of the molecular sample; and converting the string representation of the molecular sample into a molecular graph representation of the molecular sample. Further, the step of obtaining the molecular sample can also include determining the number of atoms of each atomic type in the molecule; and storing the number of atoms of each atomic type in the molecule in a digital memory. Optionally, the step of obtaining the molecular sample can include receiving a 3-Dimensional (3D) x-ray crystal structure of the molecular sample; and transforming the 3D x-ray crystal structure of the molecular sample into the string representation of the molecular sample.

Additionally, the step of pre-training the graph neural network using the masked molecular sample can include, iteratively, generating a prediction of each of the number of masked atoms using the graph neural network; and comparing each of the predicted atoms with each corresponding atom in the molecule.

The method can also include determining a total number of atoms that are being masked; and determining a number of atoms of each atomic type in the molecule that are being masked based on the determined total number of atoms that are being masked. The number of atoms of each atomic type in the molecule that are being masked can be determined based on Eq. (2).

Optionally, the step of assigning the weight to each atom in the molecule of the molecular sample can include storing each assigned weight in a digital memory. Additionally, the step of pre-training the graph neural network can include inputting the masked molecular sample into the graph neural network and performing self-supervised learning based on any one of the following self-supervised learning models: Attribute Masking (AttrMask), Graph Masked AutoEncoder (GraphMAE) or Masked Atoms Modeling (MAM). The trained graph neural network can be configured to perform prediction of an unknown molecular sample.

In an implementation, a server may be generally described as a physical device comprising at least one processor and at least one memory including computer program code. The at least one memory and the computer program code are configured to, with the at least one processor, cause the physical device to perform the requisite operations.

In conclusion, the present disclosure provides an analysis of the atom distribution of chemical molecules from a data-centric perspective. Subsequently, the present disclosure provides a simple yet effective weighted masking strategy to address the challenges arising from imbalanced and diverse atom distributions. Notably, the approach enhances the capabilities of the AttrMask task without requiring an increase in parameters, surpassing other model-centric improvement methods in terms of average performance. Moreover, it can be extended to various node-level pre-training strategies such as GraphMAE, leading to even better performance. The effectiveness of the method is substantiated by examining the trend of pre-training accuracy and provide practical guidelines for employing this method.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

What is claimed is:

1. A computer-based method of pre-training a graph neural network for molecular graphs, the method comprising:
assigning a weight to each atom in a molecule of a molecular sample based at least on a number of atoms of each atomic type in the molecule, wherein an atomic composition of the molecular sample is known;
randomly masking a number of atoms in the molecule of the molecular sample based on the assigned weight of each atom, wherein an atomic type having a higher number of atoms in the molecule has a lower probability of being masked; and
pre-training the graph neural network using the masked molecular sample.

2. The computer-based method of claim 1, wherein before assigning the weight to each atom, the method further comprises obtaining the molecular sample.

3. The computer-based method of claim 2, wherein obtaining the molecular sample comprises:
receiving a string representation of the molecular sample; and
converting the string representation of the molecular sample into a molecular graph representation of the molecular sample.

4. The computer-based method of claim 3, wherein obtaining the molecular sample further comprises:
receiving a 3-Dimensional (3D) x-ray crystal structure of the molecular sample; and
transforming the 3D x-ray crystal structure of the molecular sample into the string representation of the molecular sample.

5. The computer-based method of claim 1, wherein pre-training the graph neural network comprises:
iteratively,
generating a prediction of each of the number of masked atoms using the graph neural network; and
comparing each of the predicted atoms with each corresponding atom in the molecule.

6. The computer-based method of claim 1, wherein obtaining the molecular sample further comprises:
determining the number of atoms of each atomic type in the molecule; and
storing the number of atoms of each atomic type in the molecule in a digital memory.

7. The computer-based method of claim 1, wherein assigning the weight to each atom in the molecule of the molecular sample further comprises storing each assigned weight in a digital memory.

8. The computer-based method of claim 1, wherein the weight of each atom in the molecule of the molecular sample is determined based on the following equation:
$${w}_{a ( i )} = \frac{ln k ( {n}_{a ( i )} + 1 )}{{n}_{a ( i )}}$$
wherein wₐ₍ᵢ₎ represents the assigned weight of each atom in the molecule of the molecular sample, nₐ₍ᵢ₎ represents the number of atoms of each atomic type in the molecule, and k represents a hyperparameter.

9. The computer-based method of claim 8, wherein k is a numerical value that is more than or equal to 0.8.

10. The computer-based method of claim 1, wherein after randomly masking the number of atoms in the molecule of the molecular sample, the method further comprises:
determining a total number of atoms that are being masked; and
determining a number of atoms of each atomic type in the molecule that are being masked based on the determined total number of atoms that are being masked.

11. The computer-based method of claim 10, wherein the number of atoms of each atomic type in the molecule that are being masked is determined based on the
$${m}_{a ( i )} = M \times \frac{ln k ( {n}_{a ( i )} + 1 )}{{\sum }_{a ( i )} ln k ( {n}_{a ( i )} + 1 )}$$
wherein M represents the total number of atoms that are being masked, nₐ₍ᵢ₎ represents the number of atoms of each atomic type in the molecule, and k represents a hyperparameter.

12. The computer-based method of claim 1, wherein pre-training the graph neural network comprises inputting the masked molecular sample into the graph neural network and performing self-supervised learning based on any one of the following self-supervised learning models: Attribute Masking (AttrMask), Graph Masked AutoEncoder (GraphMAE) or Masked Atoms Modeling (MAM).

13. The computer-based method of claim 1, wherein the trained graph neural network is configured to perform prediction of an unknown molecular sample.

14. A computer-based system for pre-training a graph neural network for molecular graphs, the system comprising:
at least one processor;
a digital memory; and
a non-transitory computer-readable storage medium coupled to the at least one processor and storing programming instructions for execution by the at least one processor, the programming instructions instruct the at least one processor to perform the method according to claim 1.
